(19) United States
(12) Patent Application Publication
(10) Pub. No.: US 2006/0122436 A1
(43) Pub. Date: Jun. 8, 2006

(54) Method for producing aldehydes from alkanes

(75) Inventors: Götz-Peter Schindler, Mannheim (DE); Rocco Paciello, Bad Durkheim (DE); Klaus Harth, Hong Kong (CN)

Correspondence Address: CONNOLLY BOVE LODGE & HUTZ, LLP, P O BOX 2207, WILMINGTON, DE, 19899, US

(73) Assignee: BASF AKTIENGESELLSCHAFT Patents, Trademarks and Licenses, Ludwigshafen (DE)

(21) Appl. No.: 10/533,959

(22) PCT Filed: Nov. 3, 2003
(86) PCT No.: PCT/EP03/12201

§ 371 Date: Nov. 7, 2005

(30) Foreign Application Priority Data
Nov. 4, 2002 (DE) 102 51 262.0

(51) Int. Cl. C07C 45/50

(52) U.S. Cl. 568/429

(57) ABSTRACT

Process for preparing saturated aliphatic Cₙ-aldehydes and Cₙ₋₁-alkanes, where n is from 4 to 20, which comprises
a) providing a feed gas stream comprising one or more Cₙ₋₁-alkanes,
b) subjecting the Cₙ₋₁-alkanes to a catalytic dehydrogenation to give a product gas stream comprising unreacted Cₙ₋₁-alkanes, one or more Cₙ₋₁-alkenes and secondary constituents,
c) at least partly hydroformylating the Cₙ₋₁-alkenes in the presence of the Cₙ₋₁-alkanes and possibly the secondary constituents by means of carbon monoxide and hydrogen in the presence of a hydroformylation catalyst to give the Cₙ-aldehydes,
d) separating the product mixture obtained to give a stream comprising the Cₙ-aldehydes and a stream comprising Cₙ₋₁-alkanes and possibly secondary constituents,
e) recirculating at least part of the gas stream comprising the Cₙ₋₁-alkanes and possibly the secondary constituents as recycle gas stream to the catalytic alkane dehydrogenation (step b)).

The present invention relates to a process for preparing saturated aliphatic Cₙ-aldehydes from Cₙ₋₁-alkanes. The invention further relates to a process for the integrated preparation of saturated C₂ₙ₋₁-alcohols and C₂ₙ-alcohols from Cₙ₋₁-alkanes. In particular, the invention relates to processes of this type in which propane, butane or C₁₀-C₁₄-alkanes are used as alkanes.

The hydroformylation of olefins to produce the corresponding aldehydes is of tremendous economic importance, since the aldehydes prepared in this way are in turn starting materials for many large-volume industrial products such as solvents, plasticizer alcohols, surfactants or dispersions.

The aldehydes obtained in the hydroformylation can, for example, be hydrogenated directly to the corresponding alcohols. The aldehydes obtained can also be subjected to an aldol condensation and the condensation products obtained can subsequently be hydrogenated to give the corresponding alcohols, so that alcohols having double the number of carbon atoms are obtained.

The hydroformylation is frequently carried out as a low-pressure hydroformylation in the liquid phase using a catalyst which is homogeneously dissolved in the reaction medium, for example at from 50 to 150° C. and from 2 to 30 bar in the presence of a phosphorus-containing rhodium catalyst.

The hydroformylation of olefins is frequently carried out using olefin mixtures containing various isomers of the olefins concerned. Such olefin mixtures are obtained from steam crackers. An example is raffinate II, namely a C₄ fraction from a steam cracker which has been depleted in isobutene and butadiene.

Cracking of suitable hydrocarbons such as naphtha gives a hydrocarbon mixture which has to be subjected to a multistage work-up before the pure feed olefin for the hydroformylation is obtained. For example, propane has to be isolated from a hydrocarbon mixture comprising methane, ethane, ethene, acetylene, propane, propene, butenes, butadiene, C₅-hydrocarbons and higher hydrocarbons. The separation of propane and propene requires columns having from 10 to 100 trays. Since ethene and propene are generally obtained together in the cracking of naphtha, the amount produced of one product is always coupled to the amount produced of the other product.

If the feed olefins are not separated off from saturated hydrocarbons, the saturated hydrocarbons which are not reacted in the processes of the prior art are lost as materials of value.

It is an object of the present invention to provide a new raw materials basis for the hydroformylation of olefins. It is a further object of the invention to provide a process for the hydroformylation of olefins by means of which the hydrocarbons present in the feed gas stream to the hydroformylation are exploited very effectively.

We have found that this object is achieved by a process for preparing saturated aliphatic Cₙ-aldehydes from Cₙ₋₁-alkanes, where n is from 4 to 20, which comprises
a) providing a feed gas stream comprising one or more Cₙ₋₁-alkanes,
b) subjecting the Cₙ₋₁-alkanes to a catalytic dehydrogenation to give a product gas stream comprising unreacted Cₙ₋₁-alkanes, one or more Cₙ₋₁-alkenes and secondary constituents,
c) at least partly hydroformylating the Cₙ₋₁-alkenes in the presence of the Cₙ₋₁-alkanes and possibly the secondary constituents by means of carbon monoxide and hydrogen in the presence of a hydroformylation catalyst to give the Cₙ-aldehydes,
d) separating the product mixture obtained to give a stream comprising the Cₙ-aldehydes and a gas stream comprising Cₙ₋₁-alkanes and possibly secondary constituents,
e) recirculating at least part of the gas stream comprising the Cₙ₋₁-alkanes and possibly the secondary constituents as recycle gas stream to the catalytic alkane dehydrogenation (step b)).

Suitable alkanes which can be used in the process of the present invention have from 3 to 19 carbon atoms, preferably from 3 to 14 carbon atoms. Preference is given to propane, n-butane, isobutane, pentanes, hexanes, heptanes, octanes, nonanes, decanes, undecanes, dodecanes, tridecanes and tetradecanes as linear n-alkanes or as branched i-alkanes. Particular preference is given to propane, n-butane, isobutane and the abovementioned C₁₀-C₁₄-alkanes.

It is also possible to use mixtures of various alkanes. These mixtures can comprise isomeric alkanes having the same number of carbon atoms or alkanes having different numbers of carbon atoms. For example, a mixture of n-butane and isobutane can be used. Higher alkanes, for example the C₁₀-C₁₄-alkanes mentioned, are usually used as a mixture of alkanes having different numbers of carbon atoms, for example as a mixture of isomeric decanes, undecanes, dodecanes, tridecanes and tetradecanes.

The alkane used in the alkane dehydrogenation can further comprise secondary constituents. For example, in the case of propane, the propane used can contain up to 50% by volume of further gases such as ethane, methane, ethylene, butanes, butenes, propyne, acetylene, H₂S, SO₂ and pentanes. However, the crude propane used generally contains at least 60% by volume, preferably at least 70% by volume, particularly preferably at least 80% by volume, in particular at least 90% by volume and very particularly preferably at least 95% by volume, of propane. In the case of butane, the butane used can contain up to 10% by volume of further gases such as methane, ethane, propane, pentanes, hexanes, nitrogen and water vapor.

The alkanes mentioned can, for example, be obtained from natural gas or liquefied petroleum gas (LPG) from refineries.

Propane and butanes are preferably obtained from LPG.

The alkane or alkanes is/are partly dehydrogenated to form the corresponding alkene or alkenes. The dehydrogenation forms a product gas mixture comprising unreacted alkanes and the alkene or alkenes together with secondary constituents such as hydrogen, water, cracking products of the alkanes, CO and CO₂. The alkane dehydrogenation can be carried out with or without an oxygen-containing gas as cofeed.

The alkane dehydrogenation can in principle be carried out using all types of reactor and modes of operation known from the prior art. A comprehensive description of suitable types of reactor and modes of operation is given in “Catalytica® Studies Division, Oxidative Dehydrogenation and Alternative Dehydrogenation Processes, Study Number 4192 OD, 1993, 430 Ferguson Drive, Mountain View, Calif., 94043-5272 U.S.A.”

One suitable form of reactor is a fixed-bed tube reactor or a shell-and-tube reactor. In such a reactor, the catalyst (dehydrogenation catalyst and, when using oxygen as cofeed, a specific oxidation catalyst if appropriate) is located as a fixed bed in a reaction tube or in a bundle of reaction tubes. The reaction tubes are customarily heated indirectly by burning a gas, e.g. a hydrocarbon such as methane, in the space surrounding the reaction tubes. It is advantageous to apply this indirect form of heating only to the first about 20-30% of the length of the fixed bed and to heat the remaining length of the bed to the required reaction temperature by means of the radiant heat given off by the indirect heating. The internal diameter of the reaction tube(s) is usually from about 10 to 15 cm. A typical shell-and-tube reactor for dehydrogenation contains from about 300 to 1000 reaction tubes. The temperature in the interior of the reaction tube is usually in the range from 300 to 700° C., preferably in the range from 400 to 700° C. The reactor outlet pressure is usually from 0.5 to 8 bar, frequently from 1 to 2 bar, when using a low degree of steam dilution (corresponding to the BASF-Linde process), but can be from 3 to 8 bar when using a high degree of steam dilution (corresponding to the “steam active reforming process” (STAR process) of Phillips Petroleum Co., cf. U.S. Pat. No. 4,902,849, U.S. Pat. No. 4,996,387 and U.S. Pat. No. 5,389,342). Typical space velocities (GHSV) of propane over the catalyst are from 500 to 2000 h⁻¹. The catalyst geometry can be, for example, spherical or cylindrical (hollow or solid).

The alkane dehydrogenation can be carried out in a moving-bed reactor. For example, the moving catalyst bed can be accommodated in a radial flow reactor. In this, the catalyst slowly moves from the top downward, while the reaction gas mixture flows radially. This mode of operation is employed, for example, in the UOP Oleflex dehydrogenation process. Since the reactors in this process are operated pseudoadiabatically, it is advantageous to employ a plurality of reactors connected in series (typically up to four reactors). Upstream of or in each reactor, the gas mixture entering the reactor is heated to the required reaction temperature by combustion in the presence of added oxygen. The use of a plurality of reactors enables large differences between the temperatures of the reaction gas mixture at the reactor inlet and reactor outlet to be avoided while still achieving high total conversions. When the catalyst bed has left the moving-bed reactor, it is passed to regeneration and is subsequently reused. The dehydrogenation catalyst used generally has a spherical shape. The working pressure is typically from 2 to 5 bar. The molar ratio of hydrogen to alkane is preferably from 0.1 to 10. The reaction temperatures are preferably from 550 to 660° C.

The alkane dehydrogenation can also, as described in Chem. Eng. Sci. 1992 b, 47 (9-11) 2313, be carried out in the presence of a heterogeneous catalyst in a fluidized bed, with the alkane not being diluted. It is in this case advantageous to operate two fluidized beds in parallel, with one of these generally being in the state of regeneration. The working pressure is typically from 1 to 2 bar, and the dehydrogenation temperature is generally from 550 to 600° C. The heat required for the dehydrogenation is introduced into the reaction system by preheating the dehydrogenation catalyst to the reaction temperature. Mixing in an oxygen-containing cofeed enables the preheater to be omitted; in this case, the heat required is generated directly in the reactor system by combustion of hydrogen in the presence of oxygen. If necessary, a hydrogen-containing cofeed can additionally be mixed in.

The alkane dehydrogenation can be carried out in a tray reactor. This contains one or more successive catalyst beds. The number of catalyst beds can be from 1 to 20, advantageously from 1 to 6, preferably from 1 to 4 and in particular from 1 to 3. The reaction gas preferably flows radially or axially through the catalyst beds. In general, such a tray reactor is operated using a fixed catalyst bed. In the simplest case, the fixed catalyst beds are arranged axially in a shaft furnace reactor or in the annular gaps between concentric mesh cylinders. A shaft furnace reactor corresponds to one tray. Carrying out the dehydrogenation in a single shaft furnace reactor corresponds to a preferred embodiment. In a further preferred embodiment, the dehydrogenation is carried out in a tray reactor having three catalyst beds. In a mode of operation without oxygen as cofeed, the reaction gas mixture is subjected to intermediate heating in the tray reactor on its way from one catalyst bed to the next catalyst bed, e.g. by passing it over heat exchanger surfaces heated by means of hot gases or by passing it through tubes heated by means of hot combustion gases.

In a preferred embodiment of the process of the present invention, the alkane dehydrogenation is carried out autothermally. For this purpose, an oxygen-containing gas is additionally mixed into the reaction gas mixture of the alkane dehydrogenation in at least one reaction zone and the hydrogen present in the reaction gas mixture is burnt so that at least part of the heat of dehydrogenation required is generated directly in the reaction gas mixture in the reaction zone or zones.

In general, the amount of oxygen-containing gas added to the reaction gas mixture is selected so that combustion of the hydrogen present in the reaction gas mixture and possibly hydrocarbons present in the reaction gas mixture and/or carbon present in the form of carbon deposits generates the quantity of heat required for the dehydrogenation of the alkane to the alkene. In general, the total amount of oxygen introduced, based on the total amount of the alkane to be dehydrogenated, is from 0.001 to 0.5 mol/mol, preferably from 0.005 to 0.2 mol/mol, particularly preferably from 0.05 to 0.2 mol/mol. Oxygen can be used either as pure oxygen or as oxygen-containing gas in admixture with inert gases. The preferred oxygen-containing gas is air. The inert gases and the resulting combustion gases generally have an additional diluent effect and thus promote the heterogeneously catalyzed dehydrogenation.

The hydrogen burnt to generate heat is the hydrogen formed in the hydrocarbon dehydrogenation and, if appropriate, additional hydrogen added to the reaction gas mixture. Preference is given to adding such an amount of hydrogen that the molar ratio of H₂/O₂ in the reaction gas mixture immediately downstream of the point of introduction is from 2 to 10 mol/mol. In the case of multistage reactors, this applies to each intermediate introduction of hydrogen and oxygen.

The combustion of hydrogen occurs catalytically. The dehydrogenation catalyst used generally also catalyzes the combustion of hydrocarbons and of hydrogen in the presence of oxygen, so that in principle no other specific oxidation catalyst is required. In one embodiment, the dehydrogenation is carried out in the presence of one or more oxidation catalysts which selectively catalyze the combustion of hydrogen to oxygen in the presence of hydrocarbons. As a result, the combustion of the hydrocarbons in the presence of oxygen to form CO and CO₂ proceeds to only a subordinate extent, which has a significant positive effect on the achieved selectivities for the formation of the alkenes. The dehydrogenation catalyst and the oxidation catalyst are preferably present in different reaction zones.

In the case of a multistage reaction, the oxidation catalyst can be present in only one reaction zone, in a plurality of reaction zones or in all reaction zones.

The catalyst which selectively catalyzes the oxidation of hydrogen in the presence of hydrocarbons is preferably located in places where the oxygen partial pressures are higher than at other places in the reactor, in particular in the vicinity of the feed point for the oxygen-containing gas. Oxygen-containing gas and/or hydrogen can be introduced at one or more points on the reactor.

In one embodiment of the process of the present invention, intermediate introduction of oxygen-containing gas and of hydrogen is carried out upstream of each tray of a tray reactor. In a further embodiment of the process of the present invention, oxygen-containing gas and hydrogen are introduced upstream of each tray apart from the first tray. In one embodiment, a bed of a specific oxidation catalyst followed by a bed of the dehydrogenation catalyst is present downstream of each introduction point. In a further embodiment, no specific oxidation catalyst is present. The dehydrogenation temperature is generally from 400 to 800° C., and the outlet pressure in the last catalyst bed of the tray reactor is generally from 0.2 to 5 bar, preferably from 1 to 3 bar. The space velocity (GHSV) of propane is generally from 500 to 2000 h⁻¹, in the case of high-load operation up to 16000 h⁻¹, preferably from 4000 to 16000 h⁻¹.

The dehydrogenation can also be carried out as described in DE-A 102 11 275.

A preferred catalyst which selectively catalyzes the combustion of hydrogen comprises oxides or phosphates selected from the group consisting of the oxides and phosphates of germanium, tin, lead, arsenic, antimony and bismuth. A further preferred catalyst which catalyzes the combustion of hydrogen comprises a noble metal of transition group III or I.

The dehydrogenation catalysts used generally comprise a support and an active composition. The support usually comprises a heat-resistant oxide or mixed oxide. The dehydrogenation catalysts preferably comprise a metal oxide selected from the group consisting of zirconium dioxide, zinc oxide, aluminum oxide, silicon dioxide, titanium dioxide, magnesium oxide, lanthanum oxide, cerium oxide and mixtures thereof as support. Preferred supports are zirconium dioxide and/or silicon dioxide, and particular preference is given to mixtures of zirconium dioxide and silicon dioxide.

The active composition of the dehydrogenation catalysts generally comprises one or more elements of transition group III, preferably platinum and/or palladium, particularly preferably platinum. Furthermore, the dehydrogenation catalysts can comprise one or more elements of main groups I and/or II, preferably potassium and/or cesium. The dehydrogenation catalysts can also comprise one or more elements of main group III including the lanthanides and actinides, preferably lanthanum and/or cerium. Finally, the dehydrogenation catalysts can comprise one or more elements of main groups III and/or IV, preferably one or more elements from the group consisting of boron, gallium, silicon, germanium, tin and lead, particularly preferably tin.

In a preferred embodiment, the dehydrogenation catalyst-comprises at least one element of transition group VIII, at least one element of main groups I and/or II, at least one element of main groups III and/or IV and at least one element of transition group III including the lanthanides and actinides.

The alkane dehydrogenation is usually carried out in the presence of steam. The added steam serves as heat transfer medium and aids the gasification of organic deposits on the catalysts, so that carbonization of the catalysts is countered and the operating life of the catalyst is increased. The organic deposits are in this case converted into carbon monoxide and carbon dioxide.

The dehydrogenation catalyst can be regenerated in a manner known per se. Steam can be added to the reaction gas mixture or an oxygen-containing gas can be passed over the catalyst bed at elevated temperature from time to time so that the carbon deposits are burned off.

The alkane dehydrogenation frequently gives a mixture of isomeric alkenes. Thus, a mixture of 1-butene and 2-butene, for example in a ratio of 1:2, is obtained from n-butane. A mixture of 1-butene, 2-butene and isobutene is obtained from a mixture of n-butane and isobutane. The dehydrogenation of relatively long-chain alkanes such as the abovementioned C₁₀-C₁₄-alkanes frequently gives a mixture of all positional isomers of the corresponding alkene(s). An isomerization step can optionally follow.

The gas mixture obtained in the alkane dehydrogenation comprises the alkene or alkenes and unreacted alkanes together with secondary constituents. Usual secondary constituents are hydrogen, water, nitrogen, CO, CO₂ and cracking products of the alkanes used. The composition of the gas mixture leaving the dehydrogenation stage can vary greatly depending on the way in which the dehydrogenation is carried out. Thus, in the case of the preferred autothermal dehydrogenation with introduction of oxygen and additional hydrogen, the product gas mixture will have a comparatively high content of water and carbon oxides. In the case of modes of operation without introduction of oxygen, the product gas mixture from the dehydrogenation will have a comparatively high hydrogen content. In the case of the dehydrogenation of propane, for example, the product gas mixture leaving the dehydrogenation reactor will comprise at least the constituents propane, propene and molecular hydrogen. In addition, it will generally also contain N₂, H₂O, methane, ethane, ethylene, CO and CO₂. In the case of the dehydrogenation of butanes, the product gas mixture leaving the dehydrogenation reactor will comprise at least the constituents 1-butene, 2-butene, isobutene and hydrogen. In addition, it will generally also contain N₂, H₂O, methane, ethane, ethene, propane, propene, butadiene, CO and CO₂. The gas mixture leaving the dehydrogenation reactor will usually be at a pressure of from 0.3 to 10 bar and frequently have a temperature of from 400 to 700° C., in favorable cases from 450 to 600° C.

After the alkane dehydrogenation, unreacted Cₙ₋₁-alkanes and Cₙ₋₁-alkenes formed can be separated from secondary constituents of the product gas mixture.

Removal of water can, for example, be carried out by condensation by means of cooling and/or compression of the product gas stream from the dehydrogenation and can be carried out in one or more cooling and/or compression stages. Removal of water is usually carried out when the alkane dehydrogenation is carried out autothermally or isothermally with introduction of steam (Linde process, STAR process) and the product gas stream consequently has a high water content.

After the water has been separated off, the Cₙ₋₁-alkane(s) and the Cₙ₋₁-alkene(s) can be separated off from the remaining secondary constituents by means of a high-boiling absorption medium in an absorption/ desorption cycle. For this purpose, Cₙ₋₁-alkanes and Cₙ₋₁-alkenes are absorbed in an inert absorption medium in an absorption stage to give an absorption medium laden with Cₙ₋₁-alkanes and Cₙ₋₁-alkenes and an offgas comprising the secondary constituents, and Cₙ₋₁-alkanes and Cₙ₋₁-alkenes are liberated from the absorption medium in a desorption stage.

If alkynes, dienes and/or allenes are present in the product gas stream, their content is preferably reduced to less than 10 ppm, in particular to less than 5 ppm. This can be achieved by partial hydrogenation to the alkene, for example as described in EP-A 0 081 041 and DE-A 1 568 542.

For example, propyne or allene can be present as secondary constituents in the product gas stream from the dehydrogenation of propane. Butyne and butadiene can be present as secondary constituents in the product gas stream from butane dehydrogenation. These are preferably subjected to a partial hydrogenation to propene or butene, respectively. Catalysts suitable for the partial hydrogenation of butyne and butadiene are disclosed, for example, in WO 97/39998 and WO 97/40000.

If a catalyst which is insensitive to the alkynes, dienes and allenes mentioned is used in the subsequent hydroformylation stage, the partial hydrogenation can be omitted. Suitable catalysts are described, for example, in Johnson et al., Angewandte Chemie Int. Ed. 34 (1994), pp. 1760-61.

The Cₙ₋₁-alkanes coming from the alkane dehydrogenation are, if appropriate after removal of secondary constituents and/or partial hydrogenation, partly hydroformylated by means of carbon monoxide and hydrogen in the presence of the unreacted Cₙ₋₁-alkanes and in the presence of a hydroformylation catalyst to give the corresponding separated Cₙ-aldehydes.

This is usually done using synthesis gas, i.e. an industrial mixture of carbon monoxide and hydrogen. The hydroformylation is carried out in the presence of catalysts which are homogeneously dissolved in the reaction medium. Catalysts used are generally compounds or complexes of metals of transition group VIII, especially Co, Rh, Ir, Pd, Pt or Ru compounds or complexes, which may be unmodified or modified with, for example, amine- or phosphine-containing compounds. A review of processes carried out on an industrial scale may be found in J. Falbe, “New Synthesis with Carbon Monoxide”, Springer-Verlag 1980, page 162ff.

In the preferred embodiment of the process of the present invention in which the alkane dehydrogenation is carried out autothermally, the product gas mixture from the alkane dehydrogenation comprises alkane and alkene together with amounts of CO and H₂.

In a preferred embodiment of the process of the present invention, propene or butene are hydroformylated.

The hydroformylation of propene gives n-butyraldehyde and 2-methylpropanal. The hydroformylation of a hydrocarbon stream comprising 1-butene, 2-butene and possibly isobutene gives C₅-aldehydes, i.e. n-valeraldehyde, 2-methylbutanal and, if applicable, 3-methylbutanal. The hydroformylation of propene or butene is preferably carried out in the presence of a rhodium complex combined with a triorganophosphine ligand. The triorganophosphine ligand can be a trialkylphosphine such as tributylphosphine, an alkyldiarylphosphine such as butyldiphenylphosphine or an aryldialkylphosphine such as phenyldibutylphosphine. However, particular preference is given to triarylphosphine ligands such as triphenylphosphine, tri-p-tolylphosphine, trinaphthylphosphine, phenyldinaphthylphosphine, diphenylnaphthylphosphine, tri(p-methoxyphenyl)phosphine, tri(p-cyanophenyl)phosphine, tri(p-nitrophenyl)phosphine, p-N,N-dimethylaminophenylbisphenylphosphine and the like. Triphenylphosphine is most preferred. Propene or the butenes are partly hydroformylated. For example, it can be advantageous to carry out the butene hydroformylation under conditions under which the reaction of 1-butene occurs rapidly, while the hydroformylation of 2-butene and isobutene occurs slowly. In this way, it is possible for the hydroformylation to convert essentially only 1-butene into n-valeraldehyde and 2-methylbutanal, while the 2-butene and any isobutene remain essentially unreacted. This gives a gas stream which is depleted in butene and whose 1-butene content is reduced compared to the product gas stream from the butane dehydrogenation and which comprises essentially the original amounts of 2-butene and isobutene. The ratio of n-valeraldehyde to 2-methylbutanal in the C₅-aldehydes obtained is preferably at least 4:1, in particular at least 8:1.

The preferential hydroformylation of 1-butene compared to 2-butene and isobutene can be achieved by using a large excess of triorganophosphorus ligands and by careful control of the temperatures and the partial pressures of the reactants and/or products. Thus, the triorganophosphine ligand is preferably used in an amount of at least 100 mol per gram atom of rhodium. The temperature is preferably in the range from 80 to 130° C. and the total pressure is preferably not more than 5 000 kPa, with the partial pressure of carbon monoxide being kept below 150 kPa and the partial pressure of hydrogen being kept in the range from 100 to 800 kPa. A suitable hydroformylation process in which a mixture of butenes is used is described in EP 0 016 286.

The hydroformylation can also be carried out so that virtually complete conversion of alkenes is obtained. Suitable catalysts over which 1-butene and 2-butene are hydroformylated are, for example, the phosphite chelates described in EP-A 0 155 508 or the phosphoramidite chelates described in U.S. Pat. No. 5,710,344.

In a further preferred embodiment of the process of the present invention, C₁₀-C₁₄-alkenes are hydroformylated to give C₁₁-C₁₅-aldehydes.

While short-chain olefins are mostly hydroformylated at present using ligand-modified rhodium carbonyls as catalysts, cobalt occupies a dominant position as catalytically active central atom in the case of relatively long-chain olefins such as the C₁₀-C₁₄-alkenes. This is due firstly to the high catalytic activity of the cobalt carbonyl catalyst regardless of the position of the olefmic double bonds, the branching structure and the purity of the olefin to be reacted. Secondly, the cobalt catalyst can be separated off comparatively easily from the hydroformylation products and be returned to the hydroformylation reaction. A particularly advantageous process for the hydroformylation of C₁₀-C₁₄-alkenes comprises
I) bringing an aqueous cobalt(II) salt solution into intimate contact with hydrogen and carbon monoxide to form a hydroformylation-active cobalt catalyst, and bringing the aqueous phase comprising the cobalt catalyst into intimate contact with the C₁₀-C₁₄-alkenes and also hydrogen and carbon monoxide in at least one reaction zone so that the cobalt catalyst is extracted into the organic phase and the C₁₀-C₁₄-alkenes are hydroformylated,
II) treating the output from the reaction zone with oxygen in the presence of acidic aqueous cobalt(II) salt solution so that the cobalt catalyst is decomposed to form cobalt(II) salts and these are backextracted into the aqueous phase and subsequently separating the phases, and
III) returning the aqueous cobalt(II) salt solution to step I).

Suitable cobalt(II) salts are, in particular, cobalt carboxylates such as cobalt(II) formate, cobalt(II) acetate or cobalt ethylhexanoate and also cobalt acetylacetonate. Catalyst formation can occur simultaneously with the catalyst extraction and hydroformylation in one step in the reaction zone of the hydroformylation reactor or can be carried out in a preceding step (precarbonylation). Precarbonylation can advantageously be carried out as described in DE-A 2 139 630. The aqueous solution comprising cobalt(II) salts and cobalt catalyst obtained in this way is then introduced into the reaction zone together with the C₁₀-C₁₄-alkenes to be hydroformylated and hydrogen and carbon monoxide. However, in many cases preference is given to the formation of the cobalt catalyst, the extraction of the cobalt catalyst into the organic phase and the hydroformylation occurring in one step in which the aqueous cobalt(II) salt solution and the alkenes being brought into intimate contact with one another under hydroformylation conditions in the reaction zone. The starting materials are introduced into the reaction zone in such a way that good mixing of phases occurs and a very high phase exchange area is generated. Mixing nozzles for multiphase systems are particularly useful for this purpose.

The reactor output is depressurized after leaving the reaction zone and is passed to the cobalt removal stage. In the cobalt removal stage, the reactor output is freed of cobalt carbonyl complexes by means of air or oxygen in the presence of aqueous, weakly acidic cobalt(II) salt solution. In the cobalt removal, the hydroformylation-active cobalt catalyst is decomposed to form cobalt(II) salts. The cobalt(II) salts are backextracted into the aqueous phase. The aqueous cobalt(II) salt solution can subsequently be returned to the reaction zone or catalyst formation stage.

After the hydroformylation step, the Cₙ-aldehydes formed are separated off to give a gas stream comprising Cₙ₋₁-alkanes and unreacted Cₙ₋₁-alkenes.

The Cₙ-aldehydes formed are generally separated off by separating the hydroformylation output comprising liquid and gaseous constituents into a gas phase comprising the Cₙ-aldehydes, Cₙ₋₁-alkanes, unreacted Cₙ₋₁-alkenes, unreacted synthesis gas and possibly further incondensible constituents and a liquid phase, condensing the Cₙ-aldehydes, Cₙ₋₁-alkanes and unreacted Cₙ₋₁-alkenes from the gas phase and separating the condensate obtained into a liquid stream comprising the Cₙ-aldehydes and a gas stream comprising the Cₙ₋₁-alkanes and the unreacted Cₙ₋₁-alkenes.

The most important further incondensible constituent is nitrogen when the alkane dehydrogenation is carried out autothermally and air is used as oxygen-containing cofeed.

The separation of the hydroformylation output into a liquid phase and a gas phase is preferably carried out by
i) depressurizing the hydroformylation output comprising the liquid and gaseous constituents, which comprises the catalyst together with essentially the Cₙ-aldehyde, by-products having boiling points higher than that of the Cₙ-aldehyde, unreacted Cₙ₋₁-alkenes, Cₙ₋₁-alkanes, unreacted synthesis gas and further incondensible constituents, in a depressurization vessel,
ii) reducing the pressure and the temperature during the depressurization to such an extent that a liquid phase consisting essentially of the catalyst, by-products having boiling points higher than that of the Cₙ-aldehydes, residual amounts of Cₙ-aldehydes and unreacted Cₙ₋₁-alkenes and a gas phase consisting essentially of the Cₙ-aldehydes, unreacted Cₙ₋₁-alkenes, Cₙ₋₁-alkanes, unreacted synthesis gas and possibly further incondensible constituents are formed,
iii) taking off a liquid stream from the liquid phase obtained in this way and taking off a gaseous stream from the gas phase obtained in this way,
iv) subsequently heating the liquid stream to a temperature higher than the temperature prevailing in the depressurization vessel,
v) introducing the heated liquid stream in liquid form into the top part or the upper part of a column,
vi) introducing the gaseous stream taken off from the pressurization vessel into the bottom or the lower part of this column and conveying it in countercurrent to the liquid stream introduced in the top part or upper part of this column,
vii) taking off a gaseous stream enriched in Cₙ₋₁-alkenes and the Cₙ-aldehydes at the top of the column and passing it to further work-up,
viii) taking off a liquid stream which has a lower concentration of Cₙ-aldehydes and Cₙ₋₁-alkenes than the liquid stream introduced in the top part or upper part of the column at the bottom of this column, and
ix) recirculating all or part of this liquid stream to the hydroformylation reactor.

The essentially liquid output from the hydroformylation reactor, which generally has a temperature of from 50 to 150° C. and is under a pressure of generally from 2 to 30 bar, is depressurized in a depressurization vessel.

The liquid part of the output from the hydroformylation reaction comprises as significant constituents the catalyst, the hydroformylation product, i.e. the Cₙ-aldehyde(s) produced from the Cₙ₋₁-alkene or -alkene mixture used, by-products of the hydroformylation or solvents for the hydroformylation reaction which have boiling points higher than that of the hydroformylation product, unreacted Cₙ₋₁-alkenes and unreacted, because they are unreactive, Cₙ₋₁-alkanes.

The depressurization of the liquid hydroformylation output effects separation of the liquid hydroformylation output into a liquid phase comprising the catalyst, by-products of the hydroformylation reaction which have boiling points higher than those of the Cₙ-aldehydes, residual amounts of Cₙ₋₁-alkene and C₁-aldehydes and, if an additional high-boiling solvent has been used in the hydroformylation, this solvent and a gas phase comprising the major part of the Cₙ-aldehydes, the major part of the unreacted Cₙ₋₁-alkenes, Cₙ₋₁-alkanes and unreacted synthesis gas and also possibly further incondensible constituents.

The liquid phase separated out in the depressurization vessel is taken off from the depressurization vessel as a liquid stream and this stream is heated, for example by means of a flow-through heater or heat exchanger, to a temperature which is generally 10-80° C. above the temperature of the liquid phase in the depressurization vessel.

The liquid stream from the depressurization vessel which has been heated in this way is fed into the top part or upper part of a column which is advantageously equipped with random packing, ordered packing or internals and is conveyed in countercurrent to the gas stream which has been taken off from the upper part of the depressurization vessel and is introduced into the lower part of the column. On intimate contact of the gas stream with the heated liquid stream, the residual amounts of Cₙ-aldehydes and unreacted Cₙ₋₁-alkenes present in the liquid stream are, aided by the large surface area present in the column, transferred to the gas stream, so that the gas stream discharged at the top of the column via a line is enriched in Cₙ-aldehydes and unreacted Cₙ₋₁-alkenes while the liquid stream leaving the bottom of the column is depleted in Cₙ-aldehydes and unreacted Cₙ₋₁-alkenes.

The method of separation described is particularly advantageous because of the high alkane content of the hydroformylation output. Owing to the high content of incondensible constituents, the stripping procedure described is particularly efficient.

The liquid stream depleted in Cₙ-aldehydes and unreacted Cₙ₋₁-alkenes which leaves the column at the bottom and consists essentially of the catalyst and relatively high-boiling by-products of the hydroformylation reaction and possibly a high-boiling solvent is wholly or partly recirculated to the hydroformylation reactor.

The gas stream depleted in Cₙ-aldehydes and unreacted Cₙ₋₁-alkenes which is taken off at the top of the column and further comprises as additional constituents Cₙ₋₁-alkanes and unreacted synthesis gas is advantageously passed for the purposes of further work-up to a condenser in which the Cₙ-aldehydes, unreacted Cₙ₋₁-alkenes and Cₙ₋₁-alkanes are separated off by condensation from unreacted synthesis gas and, if applicable, the further incondensible constituents.

The unreacted synthesis gas can be recirculated to the hydroformylation reactor.

The condensible constituents separated off in the condenser, which comprise the Cₙ-aldehydes, unreacted Cₙ₋₁-alkenes and Cₙ₋₁-alkanes, are introduced into a distillation plant, which may comprise a plurality of distillation units, and separated into a stream comprising the Cₙ-aldehydes and a gas stream comprising the unreacted Cₙ₋₁-alkenes and Cₙ₋₁-alkanes. The Cₙ-aldehydes can, if appropriate after further purification, subsequently be passed to further processing to give other products of value.

The gas stream comprising the Cₙ₋₁-alkanes and possibly unreacted Cₙ₋₁-alkenes is recirculated at least in part, preferably in its entirety, as recycle gas stream to the catalytic alkane dehydrogenation (step b)). The gas recycle method achieves particularly good utilization of the hydrocarbons present in the feed gas stream to the hydroformylation, since unreacted alkanes are dehydrogenated in the dehydrogenation stage to form further alkenes and these are subsequently fed to the hydroformylation.

The Cₙ-aldehydes obtained can be subjected to an aldol condensation and the products of the aldol condensation can be catalytically hydrogenated to form C₂ₙ-alcohols.

The aldol condensation is carried out in a manner known per se, e.g. by action of an aqueous base such as sodium hydroxide solution or potassium hydroxide solution. As an alternative, it is also possible to use a heterogeneous basic catalyst such as magnesium oxide and/or aluminum oxide (cf., for example, EP-A 792 862).

The product of the aldol condensation is then catalytically hydrogenated by means of hydrogen.

Suitable hydrogenation catalysts are in general transition metals such as Cr, Mo, W, Fe, Rh, Co, Ni, Pd, Rt, Ru, etc., or mixtures thereof which can be applied to supports such as activated carbon, aluminum oxide, kieselguhr, etc., to increase the activity and stability. To increase the catalytic activity, Fe, Co and preferably Ni can also be used in the form of Raney catalysts, i.e. as metal sponge having a very high surface area. The hydrogenation conditions depend on the activity of the catalyst and the hydrogenation is preferably carried out at elevated temperatures and superatmospheric pressure. The hydrogenation temperature is preferably from about 80 to 250° C., and the pressure is preferably from about 50 to 350 bar.

The crude hydrogenation product can be worked up to give the individual alcohols by customary methods, e.g. by distillation.

In a preferred embodiment of the process of the present invention, two molecules of C₄-aldehyde are condensed to form unsaturated branched C₈-aldehydes, e.g. 2-ethylhexenal in particular, and these are hydrogenated to give the corresponding C₈-alcohols, e.g. 2-ethylhexanol in particular.

In a further preferred embodiment of the process of the present invention, two molecules of C₅-aldehyde are condensed to form unsaturated branched C₁₀-aldehydes, e.g. 2-propyl-2-heptenal and 2-propyl-4-methyl-2-hexenal in particular, and these are hydrogenated to give the corresponding C₁₀-alcohols, e.g. 2-propylheptanol and 2-propyl-4-methylhexanol in particular.

Cₙ₋₁-Alkenes which have not been reacted in the hydroformylation step can be oligomerized in the presence of the Cₙ₋₁-alkanes over an olefin oligomerization catalyst to form C₂ₙ₋₂-alkenes and these can be separated off and hydroformylated by means of carbon monoxide and hydrogen in the presence of a hydroformylation catalyst to give C₂ₙ₋₁-aldehydes. The C₂ₙ₋₁-aldehydes obtained can be catalytically hydrogenated by means of hydrogen to give the C₂ₙ₋₁-alcohols.

The present invention therefore also provides a process for the integrated preparation of saturated C₂ₙ-alcohols and C₂ₙ₋₁-alcohols from Cₙ₋₁-alkanes, where n is from 4 to 20, which comprises
a) providing a feed gas stream comprising one or more Cₙ₋₁-alkanes,
b) subjecting the Cₙ₋₁-alkanes to a catalytic dehydrogenation to give a product gas stream comprising unreacted Cₙ₋₁-alkanes, one or more Cₙ₋₁-alkenes and possibly secondary constituents,
c) partly hydroformylating the Cₙ₋₁-alkenes in the presence of the Cₙ₋₁-alkanes and the secondary constituents by means of carbon monoxide and hydrogen in the presence of a hydroformylation catalyst to give the Cₙ-aldelydes,
d) separating off the Cₙ-aldehydes formed to give, in addition, a gas stream comprising Cₙ₋₁-alkanes and unreacted Cₙ₋₁-alkenes,
e) subjecting the Cₙ-aldehydes to an aldol condensation,
f) catalytically hydrogenating the products of the aldol condensation by means of hydrogen to give the C₂ₙ-alcohols,
g) dimerizing unreacted Cₙ₋₁-alkenes in the presence of the Cₙ₋₁-alkanes and the secondary constituents over an olefin oligomerization catalyst to form C₂ₙ₋₂-alkenes and separating the product mixture obtained to give a stream comprising the C₂ₙ₋₂-alkenes and a gas stream comprising the Cₙ₋₁-alkanes and secondary constituents,
h) hydroformylating the C₂ₙ₋₂-alkenes by means of carbon monoxide and hydrogen in the presence of a hydroformylation catalyst to form C₂ₙ₋₁-aldehydes,
i) catalytically hydrogenating the C₂ₙ₋₁-aldehydes by means of hydrogen to give C₂ₙ₋₁-alcohols, and
j) recirculating at least part of the gas stream comprising the Cₙ₋₁-alkanes and secondary constituents as recycle gas stream to the alkane dehydrogenation (step b)).

If the hydroformylation step c) is carried out in such a way that the alkenes are not reacted essentially completely, further products of value can be obtained from the unreacted alkenes by dimerization, hydroformylation and hydrogenation. Thus, C₆-alkene mixtures can be obtained from unreacted propene and C₇-aldehydes such as, in particular, methylhexanals and further C₇-alcohols such as, in particular, methylhexanols can be obtained from these. In addition, the C₄-aldehydes formed in the hydroformylation step c) can be converted by aldol condensation and hydrogenation into, in particular, ethylhexanol.

In a preferred embodiment of this process, a mixture comprising butane and isobutane is catalytically dehydrogenated and, as described above, the butene hydroformylation is carried out under conditions under which the reaction of 1-butene occurs rapidly while the hydroformylation of 2-butene and isobutene occurs slowly. This gives a gas stream whose 1-butene content is reduced compared to the product gas stream of the butane dehydrogenation and which comprises essentially the original amounts of 2-butene and isobutene. 2-Butene and isobutene are oligomerized to C₈-alkenes, the product mixture obtained is fractionated, the C₈-alkenes obtained are hydroformylated to form C₉-aldehydes, in particular isononanals, and catalytically hydrogenated to give C₉-alcohols, in particular isononanols. In addition, 2-propylheptanol and 2-propyl-4-methylhexanol, in particular, are obtained from the C₅-aldehydes formed essentially from 1-butene in the hydroformylation step c) by aldol condensation and hydrogenation.

A series of processes for dimerizing lower olefins such as propene, butenes, pentenes and hexenes are known. Each of the known processes is in principle suitable for carrying out the dimerization step of the process of the present invention.

Higher olefins can be dimerized as described, for example, in WO 00/56683, WO 00/53347 and WO 00/39058.

The dimerization of olefins can be carried out in the presence of homogeneous or heterogeneous catalysts. An example of a homogeneously catalyzed process is the DIMERSOL process. In the DIMERSOL process (cf. Revue de l'Institut Franqais du Petrol, Vol. 37, No. 5, September/October 1982, page 639ff), lower olefins are dimerized in the liquid phase. Suitable precursors of the catalytically active species are, for example, (i) the system B-allylnickelphosphine/aluminum halide, (ii) Ni(O) compounds in combination with Lewis acids, e.g. Ni(COD)₂+AXₙ or Ni(CO)₂(PR₃)+AXₙ, or (iii) Ni(II) complexes in combination with alkylaluminum halides, e.g. NiX₂(PR₃)₂+Al₂Et₃Cl₃ or Ni(OCOR)₂+AlEtCl₂ (where COD=1,5-cyclooctadiene, X=Cl, Br, I; R=alkyl, phenyl; AXₙ=AlCl₃, BF₃, SbF₅ etc.). A disadvantage of homogeneously catalyzed processes is the complicated catalyst removal.

These disadvantages do not occur in heterogeneously catalyzed processes. In these processes, an olefin-containing stream is generally passed at elevated temperature over the heterogeneous catalyst in a fixed bed.

A process which is widespread in industry is the UOP process which uses H₃PO₄/SiO₂ in a fixed bed (cf., for example, U.S. Pat. No. 4,209,652, U.S. Pat. No. 4,229,586, U.S. Pat. No. 4,393,259). In the Bayer process, acidic ion exchangers are used as catalyst (cf., for example, DE 195 35 503, EP-48 893). WO 96/24567 (Exxon) describes the use of zeolites as oligomerization catalysts. Ion exchangers such as Amberlite are also used in the process of Texas Petrochemicals (cf. DE 3 140 153).

It is also known that lower olefins can be dimerized in the presence of alkali metal catalysts (cf. Catalysis Today, 1990, 6, p. 329ff).

For the present purposes, preference is given to carrying out the alkene dimerization over a heterogeneous nickel-containing catalyst. Suitable heterogeneous, nickel-containing catalysts can have different structures, with catalysts comprising nickel oxide being preferred. It is possible to use catalysts which are known per se, as are described in C. T. O'Connor et al., Catalysis Today, Volume 6 (1990), pages 336-338. In particular, use is made of supported nickel catalysts. The support materials can be, for example, silica, alumina, aluminosilicates, aluminosilicates having layer structures and zeolites, zirconium oxide which may have been treated with acids or sulfated titanium dioxide. Precipitated catalysts which can be obtained by mixing aqueous solutions of nickel salts and silicates, e.g. sodium silicate with nickel nitrate, and, if desired, aluminum salts such as aluminum nitrate and calcining the precipitate are particularly useful. It is also possible to use catalysts which are obtained by incorporation of Ni²⁺ ions into natural or synthetic sheet silicates such as montmorillonites by ion exchange. Suitable catalysts can also be obtained by impregnation of silica, alumina or aluminosilicates with aqueous solutions of soluble nickel salts such as nickel nitrate, nickel sulfate or nickel chloride and subsequent calcination.

Particular preference is given to catalysts which consist essentially of NiO, SiO₂, TiO₂ and/or ZrO₂ and, if desired, Al₂O₃. They lead to dimerization occurring preferentially over the formation of higher oligomers and give predominantly linear products. A catalyst comprising as significant active constituents from 10 to 70% by weight of nickel oxide, from 5 to 30% by weight of titanium dioxide and/or zirconium dioxide, from 0 to 20% by weight of aluminum oxide and silicon dioxide as balance is most preferred. Such a catalyst is obtainable by precipitation of the catalyst composition at pH 5-9 by addition of an aqueous solution containing nickel nitrate to an alkali metal water glass solution containing titanium dioxide and/or zirconium dioxide, filtration, drying and heat treatment at from 350 to 650° C. Specific reference may be made to DE 4 339 713 for the preparation of these catalysts. The entire disclosure of this document and the prior art cited therein is hereby incorporated by reference.

The catalyst is preferably in shaped or pelletized form, e.g. in the form of pellets, e.g. pellets having a diameter of from 2 to 6 mm and a height of from 3 to 5 mm, rings having, for example, an external diameter of from 5 to 7 mm, a height of from 2 to 5 mm and a hole diameter of from 2 to 3 mm or extrudates of various lengths having a diameter of, for example, from 1.5 to 5 mm. Such shapes are obtained in a manner known per se by tableting or extrusion, usually with use of a catalytic aid such as graphite or stearic acid.

Dimerization over heterogeneous, nickel-containing catalysts is usually carried out at from 30 to 280° C., preferably from 30 to 140° C. and particularly preferably from 40 to 130° C. It is preferably carried out at a pressure of from 10 to 300 bar, in particular from 15 to 100 bar and particularly preferably from 20 to 80 bar. The pressure is advantageously set so that the hydrocarbon stream is liquid or in a supercritical state at the temperature selected.

The gas stream comprising the Cₙ₋₁-alkanes and Cₙ₋₁-alkenes is advantageously passed over one or more fixed-bed catalysts. Suitable reaction apparatuses for bringing the gas stream into contact with the heterogeneous catalyst are known to those skilled in the art. Examples of suitable apparatuses are shell-and-tube reactors or shaft ovens. Owing to the lower capital costs, shaft ovens are preferred. The dimerization can be carried out in a single reactor in which the oligomerization catalyst may be present in a single fixed bed or a plurality of fixed beds. As an alternative, a reactor cascade comprising a plurality of reactors, preferably two reactors, connected in series can be used for carrying the oligomerization, with the dimerization being carried out to only a partial conversion during passage through the reactor or reactors located upstream of the last reactor of the cascade and the desired final conversion being achieved only when the reaction mixture passes through the last reactor of the cascade.

The hydroformylation of the C₂ₙ₋₂-alkenes to C₂ₙ₋₁-aldehydes which follows the dimerization can be carried out as described above. The C₂ₙ₋₁-aldehydes can also be separated off as described above.

The catalytic hydrogenation of the C₂ₙ₋₁-aldehydes to give the C₂ₙ₋₁-alcohols can be carried out as described above in the context of the hydrogenation of the aldol condensation products.

In a further embodiment of the process of the present invention, the hydroformylation of the C₂ₙ₋₂-alkenes to form the C₂ₙ₋₁-aldehydes and the hydrogenation to give the C₂ₙ₋₁-alcohols is carried out in one step without isolation of the aldehydes.

A gas stream comprising the Cₙ₋₁-alkanes, possibly unreacted Cₙ₋₁-alkenes and secondary constituents is obtained and this is recirculated at least in part, preferably in its entirety, as recycle gas stream to the alkane dehydrogenation (step b)). The gas recycle mode achieves particularly good utilization of the hydrocarbons present in the feed gas stream to the process.

However, the hydroformylation step and the aldol condensation step can also be omitted. Accordingly, the present invention also provides a process for the integrated preparation of saturated C₂ₙ₋₁-alcohols from Cₙ₋₁-alkanes, where n is from 4 to 20, which comprises
a) providing a feed gas stream comprising one or more Cₙ₋₁-alkanes,
b) subjecting the Cₙ₋₁-alkanes to a catalytic dehydrogenation to give a product gas stream comprising unreacted Cₙ₋₁-alkanes, one or more Cₙ₋₁-alkenes and possibly secondary constituents,
c) dimerizing the Cₙ₋₁-alkenes in the presence of the Cₙ₋₁-alkanes and the secondary constituents over an olefin oligomerization catalyst to form C_{2n 2}-alkenes and separating the product mixture obtained to give a stream comprising the C₂ₙ₋₂-alkenes and a gas stream comprising the Cₙ₋₁-alkanes and secondary constituents,
d) hydroformylating the C₂ₙ₋₂-alkenes by means of carbon monoxide and hydrogen in the presence of a hydroformylation catalyst to form C₂ₙ₋₁-aldehydes,
e) catalytically hydrogenating the C₂ₙ₋₁-aldehydes by means of hydrogen to give C₂ₙ₋₁-alcohols, and
f) recirculating at least part of the gas stream comprising the Cₙ₋₁-alkanes and secondary constituents as recycle gas stream to the alkane dehydrogenation (step b)).

We claim:

1. The process for preparing saturated aliphatic Cₙ-aldehydes from Cₙ₋₁-alkanes, where n is from 4 to 20, which comprises:
a) providing a feed gas stream comprising one or more Cₙ₋₁-alkanes;
b) subjecting the Cₙ₋₁-alkanes to a catalytic dehydrogenation to give a product gas stream comprising unreacted Cₙ₋₁-alkanes, one or more Cₙ₋₁-alkenes and secondary constituents;
c) at least partly hydroformylating the Cₙ₋₁-alkenes in the presence of the Cₙ₋₁-alkanes and possibly the secondary constituents by means of carbon monoxide and hydrogen in the presence of a hydroformylation catalyst to give the Cₙ-aldehydes;
d) separating the product mixture obtained to give a stream comprising the Cₙ-aldehydes and a stream comprising Cₙ₋₁-alkanes and possibly secondary constituents; and
e) recirculating at least part of the gas stream comprising the Cₙ₋₁-alkanes and possibly the secondary constituents as recycle gas stream to the catalytic alkane dehydrogenation (step b)).

2. The process according to claim 1 for preparing C₄-aldehydes from propane.

3. The process according to claim 1 for preparing C₅-aldehydes from butane.

4. The process according to claim 1 for preparing saturated aliphatic C₁₁-C₁₅-aldehydes from C₁₀-C₁₄-alkanes.

5. The process according to claim 1 wherein the catalytic alkane dehydrogenation (step b)) is carried out autothermally.

6. The process for preparing saturated aliphatic C₂ₙ-alcohols from Cₙ₋₁-alkanes, which comprises carrying out the steps a) to e) as defined in claim 1 and additionally
f) subjecting the Cₙ-aldehydes to an aldol condensation, and
g) catalytically hydrogenating the products of the aldol condensation by means of hydrogen to give the C₂ₙ-alcohols.

7. The process for the integrated preparation of saturated C₂ₙ₋₁-alcohols and C₂ₙ-alcohols from Cₙ₋₁-alkanes, where n is from 4 to 20, which comprises:
a) providing a feed gas stream comprising one or more Cₙ₋₁-alkanes;
b) subjecting the Cₙ₋₁-alkanes to a catalytic dehydrogenation to give a product gas stream comprising unreacted Cₙ₋₁-alkanes, one or more Cₙ₋₁-alkenes and possibly secondary constituents;
c) partly hydroformylating the Cₙ₋₁-alkenes in the presence of the Cₙ₋₁-alkanes and the secondary constituents by means of carbon monoxide and hydrogen in the presence of a hydroformylation catalyst to give the Cₙ-aldelydes;
d) separating off the Cₙ-aldehydes formed to give, in addition, a gas stream comprising Cₙ₋₁-alkanes and unreacted Cₙ₋₁-alkenes;
e) subjecting the Cₙ-aldehydes to an aldol condensation;
f) catalytically hydrogenating the products of the aldol condensation by means of hydrogen to give the C₂ₙ-alcohols;
g) dimerizing unreacted Cₙ₋₁-alkenes in the presence of the Cₙ₋₁-alkanes and the secondary constituents over an olefin oligomerization catalyst to form C₂ₙ₋₂-alkenes and separating the product mixture obtained to give a stream comprising the C₂ₙ₋₂-alkenes and a gas stream comprising the Cₙ₋₁-alkanes and secondary constituents;
h) hydroformylating the C₂ₙ₋₂-alkenes by means of carbon monoxide and hydrogen in the presence of a hydroformylation catalyst to form C₂ₙ₋₁-aldehydes;
i) catalytically hydrogenating the C₂ₙ₋₁-aldehydes by means of hydrogen to give C₂ₙ₋₁-alcohols; and
j) recirculating at least part of the gas stream comprising the Cₙ₋₁-alkanes and secondary constituents as recycle gas stream to the alkane dehydrogenation (step b)).

8. The process according to claim 7 for the integrated preparation of saturated C₇-alcohols and C₈-alcohols from propane.

9. The process according to claim 8 for the integrated preparation of saturated C₉-alcohols and C₁₀-alcohols from butane.

10. The process according to claim 7, wherein the catalytic alkane dehydrogenation (step b)) is carried out autothermally.

11. The process for the integrated preparation of saturated C₂ₙ₋₁-alcohols from Cₙ₋₁-alkanes, where n is from 4 to 20, which comprises:
a) providing a feed gas stream comprising one or more Cₙ₋₁-alkanes;
b) subjecting the Cₙ₋₁-alkanes to a catalytic dehydrogenation to give a product gas stream comprising unreacted Cₙ₋₁-alkanes, one or more Cₙ₋₁-alkanes and possibly secondary constituents;
c) dimerizing Cₙ₋₁-alkenes in the presence of the Cₙ₋₁-alkanes and the secondary constituents over an olefin oligomerization catalyst to form C₂ₙ₋₂-alkenes and separating the product mixture obtained to give a stream comprising the C₂ₙ₋₂-alkenes and a gas stream comprising the Cₙ₋₁-alkanes and secondary constituents;
d) hydroformylating the C₂ₙ₋₂-alkenes by means of carbon monoxide and hydrogen in the presence of a hydroformylation catalyst to form C₂ₙ₋₁-aldehydes;
e) catalytically hydrogenating the C₂ₙ₋₁-aldehydes by means of hydrogen to give C₂ₙ₋₁-alcohols; and
f) recirculating at least part of the gas stream comprising the Cₙ₋₁-alkanes and secondary constituents as recycle gas stream to the alkane dehydrogenation (step b)).

12. The process according to claim 2, wherein the catalytic alkane dehydrogenation (step b)) is carried out autothermally.

13. The process according to claim 3, wherein the catalytic alkane dehydrogenation (step b)) is carried out autothermally.

14. The process according to claim 4, wherein the catalytic alkane dehydrogenation (step b)) is carried out autothermally.

15. The process according to claim 8, wherein the catalytic alkane dehydrogenation (step b)) is carried out autothermally.

16. The process according to claim 9, wherein the catalytic alkane dehydrogenation (step b)) is carried out autothermally.
